(19) United States
(12) Patent Application Publication
(10) Pub. No.: US 2026/0356900 A1
(43) Pub. Date: Nov. 12, 2026

(54) Wnt7 Molecules with Amino Acid Addition

(71) Applicant: NEUVASQ BIOTECHNOLOGIES SA, Gosselies (BE)

(72) Inventors: Benoit VANHOLLEBEKE, Charleroi (BE); Emilien NICOLAS, Charleroi (BE); Isabel VOGEL, Charleroi (BE)

(21) Appl. No.: 19/488,379

(22) PCT Filed: May 30, 2024
(86) PCT No.: PCT/EP2024/064864

§ 371 Date: Nov. 26, 2025

(30) Foreign Application Priority Data
May 30, 2023 (EP) 23176176.8

(51) Int. Cl. C07K 14/475; A61K 38/00

(52) U.S. Cl. CPC C07K 14/475; A61K 38/00

(57) ABSTRACT

The current invention relates to a Wnt7 protein comprising at its C-terminal end an amino acid addition, having a length of 1 to 3 amino acids. The invention further relates to a nucleic acids encoding the Wnt7 protein, a nucleic acid expression cassette, a vector, and a pharmaceutical composition; and their uses in medicine and therapy.

### FIELD OF THE INVENTION

The present invention is directed to Wnt7a/b proteins having an amino acid addition at their C-terminal end, in a length of 1 to 3 amino acids. The invention also relates to nucleic acids encoding such proteins and to their use in medicine.

### BACKGROUND

Endothelial Wnt/β-catenin signaling acts as a master regulator of Blood-Brain Barrier (BBB) physiology in response to neural-derived Wnt7a/b ligands. Wnt/β-catenin signaling initiates the BBB differentiation cascade at the earliest steps of central nervous system (CNS) vascular invasion, and then maintains BBB function in adults. Recent evidence suggests that inhibition of Wnt signaling by conditional deletion of β-catenin signaling in cerebral endothelial cells (ECs) causes BBB breakdown and accelerates disease progression in stroke, glioblastoma, and multiple sclerosis murine models. Conversely, recombining a constitutively active form of β-catenin in the CNS endothelium is protective in models of brain cancer and stroke.

Wnt7a/b being the endogenous ligands controlling β-catenin-dependent BBB maturation, they constitute, in principle, legitimate therapeutic agents to repair the dysfunctional BBB. However, activation of Wnt signaling via Wnt7a/b ligands has pleiotropic outcomes across a range of tissues and organs, in both health and disease. More so, the structural modalities of Wnt/Frizzled (FZD) interactions disqualify natural Wnt ligands as safe therapeutics. On the other hand, it is known that Wnt7a/b ligands activate Wnt signaling via two distinct types of membrane receptor complexes. The first one is the Frizzled (FZD) family receptors/LRP5/6 complex which has broad tissue distribution, binds non-discriminately to Wnt7a/b and other Wnt ligands and leads to systemic activation of Wnt signaling, the second one is the GPR124/RECK/FZ/LRP5/6 complex which is enriched at the level of cerebral endothelial cells where GPR124 and RECK are expressed.

Recently, proof-of-concept mutagenesis has revealed that Wnt7a ligands can be engineered into highly-specific GPR124/RECK agonists to induce Wnt signaling in cells expressing GPR124 and RECK such as in cerebral endothelial cells (“on-target”) and do not induce Wnt signaling in the absence of GPR124 and/or RECK (“off-target”). Results suggest a promising use of engineered Wnt7a ligands as therapeutics, especially for CNS related disorders.

WO2019180204 and Martin et al., 2022, describe Wnt7a/b variants which activate GPR124/RECK/Frizzled/LRP-mediated Wnt signaling with impaired activation of Frizzled/LRP-mediated Wnt signaling in the absence of RECK and/or GPR124, the latter which would lead to “off-target” activity. To be used in therapy, unwanted “off-target” activity should be as low as possible, especially when targeting a pathway such as Wnt signaling which has broad functions across the developmental stages and which deregulation is known to be implicated in many human diseases, ranging from cancers to skeletal disorders. Although the previously discovered Wnt7a/b variants showed low “off-target” activity, considering the broad role of WNT signaling in normal development and disease-related processes, there is still a need for additional GPR124/RECK/Frizzled/LRP-mediated Wnt signaling specific agonists with optimal off-target effect.

The present invention aims to resolve at least some of the problems and disadvantages mentioned above. The invention thereto aims to provide further novel Wnt7a or Wnt7b derived GPR124/RECK co-receptor complex specific agonists.

### SUMMARY OF THE INVENTION

The present invention and embodiments thereof serve to provide a solution to one or more of above-mentioned disadvantages. To this end, the present invention relates to a Wnt7a/b protein according to claim 1. The protein as disclosed herein comprises at its C-terminal end an amino acid addition which can have a length of maximum 3 amino acids.

The Wnt7a/b proteins disclosed herein are preferably agonists of G-protein coupled receptor GPR124/RECK/Frizzled/lipoprotein receptor-related protein (LRP) complex. Said Wnt7a/b proteins as disclosed herein are not able to or have an impaired capacity to bind to Frizzled and/or lipoprotein receptor-related protein (LRP) in the absence of RECK and/or GPR124; and/or said Wnt7a/b proteins are not able to or have an impaired capacity to activate Frizzled and/or lipoprotein receptor-related protein (LRP) in the absence of RECK and/or GPR124.

It was shown that such Wnt7a/b proteins are particularly efficient agonists of GPR124/RECK specific signaling, while not or only limited activating canonical Wnt signaling via Frizzled/LPR in the absence of GPR124 and/or RECK. Wnt proteins exert pleiotropic functions by regulating cell proliferation, differentiation, migration, apoptosis, polarity and genetic stability. Dysregulated Wnt signaling levels are associated with a large spectrum of human pathologies. Intervening in Wnt signaling pathways generally leads to multiple effects, of which many were unwanted (“off-target”).

As the GPR124/RECK co-receptor complex is enriched at the level of the blood brain barrier endothelial cells, said Wnt proteins of the present invention can selectively (re-)activate Wnt signaling in cerebral endothelial cells (“on-target”), without activating unwanted “off-target” Wnt-related pathways.

Preferred embodiments of the protein of claim 1 are shown in any of the claims 2 to 7.

In other aspects, the present invention relates to a nucleic acid encoding the protein, a nucleic acid expression cassette comprising said nucleic acid, and a vector comprising said nucleic acid or the nucleic acid expression cassette according to claims 8, 9 and 10, respectively.

In a further aspects, the present invention relates to a pharmaceutical composition according to claim 11, the use of the Wnt7a/b protein, the nucleic acid, the nucleic acid expression cassette, the vector or the pharmaceutical composition as a medicament according to claim 12 to treat and/or prevent disorders, preferably of a neurovascular disorder or a central nervous system (CNS) disorder comprising neurovascular dysfunction.

In another aspect, the invention relates to the nucleic acid, the nucleic acid expression cassette, or the vector, for use in gene therapy according to claim 13.

In a final aspect, the invention relates to the nucleic acid, or the vector, for use in RNA therapy according to claim 14.

### DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

FIG. 1 shows the effect of the addition of 1, 2, or to 5 amino acids to the C-terminal end of wildtype mouse Wnt7a, on “on-target” activation of the canonical Wnt pathway with GPR124/RECK (on-target) and “off-target” activation of the canonical pathway without GPR124/RECK (off-target). The resulting constructs (FIG. 1A) were transfected in HEK293 STF cells together with Renilla luciferase, and with Lrp5, Fz1 receptor, Gpr124, and Reck constructs (On-target), or with Renilla luciferase, and with Lrp5 and Fz5 receptor (off target) and tested in a dual luciferase assay. On-target activity of the wt Wnt7a is maintained even after the addition of 1 to 5 amino acids at the C-terminus, while off-target activity is significantly reduced. (FIG. 1B).

FIG. 2 shows the effect of the addition of each of the 20 natural amino acids on the signaling properties of wild-type Wnt7a. Interestingly, the addition of certain amino acids to the C-terminal end of the wild-type mWnt7a protein significantly decreases its off-target activity. The addition of the Methionine residue is particularly interesting because this addition keeps an on-target activity similar to mWnt7a without residue addition but leads to an undetectable off-target activity using the in vitro luciferase assay. This C-terminal addition of Methionine to the wild-type Wnt7a protein thus yields a promising variant for clinical studies.

### DETAILED DESCRIPTION OF THE INVENTION

The present invention concerns Wnt7a/b proteins with amino acid addition up to 3 amino acid in length. The proteins are specific agonists of the G-protein coupled receptor GPR124 and RECK-dependent Wnt/β-catenin signaling pathway, a major regulator of blood brain barrier (BBB). The invention thereto relates to Wnt7a/b proteins that are agonists of a G-protein coupled receptor (GPR)124/RECK/Frizzled/lipoprotein receptor-related protein (LRP) complex, and are not able to or have an impaired capacity to bind to or to activate Frizzled and/or lipoprotein receptor-related protein (LRP) in the absence of RECK and/or GPR124.

The invention in particular concerns Wnt7a/b proteins, having at their C-terminal end an amino acid addition, having a length of 1 to 3 amino acids.

The present invention further concerns the nucleic acids and vectors encoding said Wnt7a/b proteins and pharmaceutical compositions comprising said protein, nucleic acid and/or vector, and their uses in medicine.

### DEFINITIONS

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

A peptide, polypeptide or protein can be naturally occurring, e.g., present in or isolated from nature, e.g., produced or expressed natively or endogenously by a cell or tissue and optionally isolated therefrom. A peptide, polypeptide or protein can be recombinant, i.e., produced by recombinant DNA technology, and/or can be, partly or entirely, chemically or biochemically synthesized. Without limitation, a peptide, polypeptide or protein can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis.

The term “biologically active” is interchangeable with terms such as “functionally active” or “functional”, denoting that the proteins disclosed herein at least partly retains the biological activity or intended functionality of the respective or corresponding peptide, polypeptide or protein. Reference to the “activity” of a protein may generally encompass any one or more aspects of the biological activity of the protein, such as without limitation any one or more aspects of its biochemical activity, enzymatic activity, signaling activity, interaction activity, ligand activity, and/or structural activity, e.g., within a cell, tissue, organ or an organism. 35 Preferably, a functionally active protein may retain at least about 20%, e.g., at least about 25%, or at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% of the intended biological activity or functionality compared with the corresponding protein.

In certain embodiments, a functionally active protein may even display higher biological activity or functionality compared with the corresponding protein, for example may display at least about 100%, or at least about 150%, or at least about 200%, or at least about 300%, or at least about 400%, or at least about 500% of the intended biological activity or functionality compared with the corresponding protein. By means of an example, where the activity of a given peptide, polypeptide or protein can be readily measured in an assay with a quantitative output, for example an enzymatic assay or a signaling assay or a binding assay producing a quantifiable signal, a functionally active protein may produce a signal which is at least about 20%, or at least about 25%, or at least 30%, or at least about 40%, or at least about 50%, or at least 60%, more preferably at least about 70%, or at least 80%, or at least about 85%, or at least about 90%, or at least about 95%, or at least about 100%, or at least about 150%, or at least about 200%, or at least about 300%, or at least about 400%, or at least about 500% of the signal produced by the corresponding protein.

“Wnt7” in the present context refers to both “Wnt7a” and/or “Wnt7b”, which are part of the Wnt family of proteins. The terms “Wnt7” and “WNT7” are used interchangeably throughout this specification.

A “GPR124/RECK/FZD/LRP receptor complex”, “GPR124/RECK/FZD/LRP co-receptor complex”, “GPR124/RECK/FZD/LRP complex” or “GPR124/RECK/FZD/LRP protein complex” broadly denotes a protein complex, particularly a membrane-associated protein complex, more particularly a plasma membrane-associated protein complex comprising at least one GPR124 protein, at least one RECK protein, at least one FZD protein and at least one LRP protein. A GPR124/RECK/FZD/LRP co-receptor complex, when located at the plasma membrane of a cell, is capable of activating Wnt/B-catenin signaling in said cell in response to extracellularly provided Wnt7 ligand.

A “FZD/LRP receptor complex”, “FZD/LRP co-receptor complex”, “FZD/LRP complex” or “FZD/LRP protein complex” broadly denotes a protein complex, particularly a membrane-associated protein complex, more particularly a plasma membrane associated protein complex comprising at least one FZD protein and at least one LRP protein. A FZD/LRP receptor complex, when located at the plasma membrane of a cell, is capable of activating Wnt/β-catenin signalling in said cell in response to extracellularly provided Wnt ligand, such as, but not limited to Wnt7 ligand.

“LRP” or “lipoprotein receptor-related protein” encompasses any and all lipoprotein receptor-related proteins, also known in the art as low-density lipoprotein receptor related proteins or prolow-density lipoprotein receptor-related proteins. In certain particularly preferred embodiments, the terms denote LRP5, LRP6, or LRP5 and LRP6 (LRP5/6).

The terms “bind”, “interact”, “specifically bind” or “specifically interact” as used throughout this specification mean that an agent binds to or influences one or more desired molecules or analytes substantially to the exclusion of other molecules which are random or unrelated, and optionally substantially to the exclusion of other molecules that are structurally related. The terms do not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, such as, e.g., at least about 1000-fold or more greater, at least about 1×10⁴-fold or more greater, or at least about 1×10⁵-fold or more greater, than its affinity for a non-target molecule.

The term “amino acid addition” as used throughout this specification means an entity of one to three amino acids which can be added to C-terminal end of a protein or a (poly)peptide, preferably via peptide bonds. The term may be used to refer only one, two or three amino acids. If longer than one amino acid, the amino acids of the amino acid addition are preferably joined together by peptide bonds.

The term “gene therapy” and/or “RNA therapy” as used herein refers to the introduction of an exogenous polynucleotide into a host cell for therapeutic or prophylactic purposes, irrespective of the method used for the introduction. Such methods include a variety of well-known techniques such as vector-mediated gene transfer (by, e.g., viral infection/transfection, or various other protein-based or lipid-based gene delivery complexes) as described elsewhere herein. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art. The RNA molecule may be any type of RNA molecule. For instance, the RNA molecule may be cytoplasmic RNA, a nuclear RNA, an mRNA, antisense RNA or a noncoding RNA, preferably an mRNA.

The terms “host cell” and “host organism” may suitably refer to cells or organisms encompassing both prokaryotes, such as bacteria, and eukaryotes, such as yeast, fungi, protozoan, plants and animals. Contemplated as host cells are inter alia unicellular organisms, such as bacteria (e.g., E. coli, Salmonella typhimurium, Serratia marcescens, or Bacillus subtilis), yeast (e.g., Saccharomyces cerevisiae or Pichia pastoris), (cultured) plant cells (e.g., from Arabidopsis thaliana or Nicotiana tobaccum) and (cultured) animal cells (e.g., vertebrate animal cells, mammalian cells, primate cells, human cells or insect cells). Contemplated as host organisms are inter alia multi-cellular organisms, such as plants and animals, preferably animals, more preferably warm-blooded animals, even more preferably vertebrate animals, still more preferably mammals, yet more preferably primates; particularly contemplated are such animals and animal categories which are non-human.

The phrases “central nervous system (CNS) disorder comprising neurovascular dysfunction” or “CNS disease comprising neurovascular dysfunction” as used herein refer to a group of neurological disorders that affect the structure and/or function of the brain and/or spinal cord, which collectively form the CNS, that are caused by, contributed to, or typified by neurovascular dysfunction, or which lead to an abnormality of the structure and/or function of the blood vessels within or supplying the brain and/or spine. Non-limiting examples of central nervous system (CNS) disorders comprising neurovascular dysfunction are multiple sclerosis, ischemic stroke, brain cancer, glioblastoma, epilepsy, dementia, vascular dementia, HIV-1-associated dementia, Alzheimer's disease, Parkinson's disease, Huntington disease, amyotrophic lateral sclerosis, infectious brain diseases, traumatic brain injuries, migraine, chronic traumatic encephalopathy and retinal vascular disorders, including but not limited to Norrie disease, familial exudative vitreoretinopathy, osteoporosis-pseudoglioma syndrome, diabetic retinopathy and macular degeneration.

The terms “neurovascular disorder” or “neurovascular disease” as used herein refer to a disease or pathological alteration or condition which affects the cerebral vascular system and/or the vascular system supplying the spinal cord. The terms encompass any abnormality of the blood vessels within or supplying the brain and/or spine. Abnormalities may be without limitation (i) narrowing of arteries which reduces blood flow to the brain that may lead to hypoxia, ischemia or stroke and/or (ii) weakening of arteries that may lead to brain aneurysms and increase the risk of intracranial bleeding. Non-limiting examples of neurovascular diseases are ischemic stroke, hemorrhagic stroke, ischemia/reperfusion injury, brain aneurysms, arteriovenous malformations (AVMs), cavernous malformations, vasculitis, cerebral hemorrhage, subarachnoid hemorrhage, spinal vascular malformations, carotid artery stenosis, Moyamoya disease, intracranial atherosclerosis and retinal vascular disorders, including but not limited to Norrie disease, familial exudative vitreoretinopathy, osteoporosis-pseudoglioma syndrome, diabetic retinopathy and macular degeneration.

Except when noted, the terms “subject” or “patient” can be used interchangeably and refer to animals, preferably warm-blooded animals, more preferably vertebrates, even more preferably mammals, still more preferably primates, and specifically includes human patients and non-human mammals and primates. Preferred subjects are human subjects. The terms “subject” or “patient” include subjects in need of treatment, more particularly subjects that would benefit from treatment of a given condition, particularly a neurovascular disorder or a central nervous system (CNS) disorder comprising neurovascular dysfunction. Such subjects may include, without limitation, those that have been diagnosed with said condition, those prone to develop said condition and/or those in who said condition is to be prevented.

The term “therapeutically active/effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated. The term “prophylactically active/effective amount” refers to an amount of an active compound or pharmaceutical agent that inhibits or delays in a subject the onset of a disorder as being sought by a researcher, veterinarian, medical doctor or other clinician. In present context, the “therapeutically active amount” is used to refer to both the “therapeutically active amount” and “prophylactically active amount” as described above, unless the distinction is clear from the context. Methods are known in the art for determining therapeutically and/or prophylactically effective doses of a compound, a protein, a nucleic acid encoding the compound/protein, a nucleic acid expression cassette, or a pharmaceutical composition, as taught herein. The term “therapeutically effective dose” as used herein refers to an amount of a compound, a protein, a nucleic acid encoding the compound/protein, a nucleic acid expression cassette, or a pharmaceutical composition, as taught herein, that when administered brings about a positive therapeutic response with respect to treatment of a patient having a specific disease or disorder.

### Description

### Compounds

Previously the inventors characterized the first-ever “Wnt decoding module” capable of discriminating Wnt ligands that are otherwise largely synonymous in their capacity to bind Frizzled, thereby helping cells interpret the complexity of Wnt signaling inputs in order to orchestrate tissue development and homeostasis. In the herein characterized Wnt decoding module, selectivity is conferred by RECK, which mediates Wnt7-specific binding in a Frizzled-independent manner and is part of Wnt7 ligand-specific RECK/GPR124/Frizzled/lipoprotein receptor-related protein (LRP) signalosomes.

The inventors further demonstrated the design of agonists capable of activating Wnt signaling selectively in cells expressing RECK and GPR124 i.e. for instance cerebral endothelial cells. They further showed that such agonists are useful as therapeutics, such as particularly for the treatment of neurovascular disorders or central nervous system (CNS) disorders comprising neurovascular dysfunction. Hence, the invention allowed to provide inter alia agonists capable of stimulating Wnt/β-catenin signaling in cerebral endothelial cells (“on-target activity”) with only low cross-reactivity with other Frizzled pathways (“unwanted” or “off-target activity”), and useful as therapeutics, particularly for neurovascular disorders or central nervous system (CNS) disorders comprising neurovascular dysfunction.

Herein disclosed is a surprising discovery that said Wnt7 wild-type proteins with additional amino acids displaying highly specific “on-target” GPR124/RECK activity, but limited the “off-target” activity, preferably undetectable and thus (almost) reduced to 0. Such proteins are agonists of a GPR124/RECK co-receptor complex, and are preferably not able to activate Frizzled and/or LRP in the absence of RECK and/or GPR124. Said proteins further comprise a wild-type Wnt7 without any modification in its sequence except an additional one to three amino acids at its C-terminal end.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

In a first aspect, the invention provides a Wnt7a/b protein, wherein said protein comprises at its C-terminal end an amino acid addition, having a length of maximum 3 amino acids.

In embodiments, the Wnt7 protein disclosed herein is an agonist, preferably a selective agonist, of the Gpr124- and Reck-dependent Wnt/β-catenin pathway.

In an embodiment, the term “selective agonist of the Gpr124- and Reck-dependent Wnt/β-catenin pathway” is to be understood as an agonist of the said pathway that is able to activate said pathway with high selectivity and specificity (on-target activity), with no or only minimal activation of other Wnt signaling pathways (off-target activity). In embodiments, the Wnt7a/b protein disclosed herein as an agonist of Gpr124- and Reck-dependent Wnt/β-catenin pathway shows less than 20% off-target activity, for example, showed less than 15%, 10%, 5% or 1% off-target activity, preferably less than 10% off-target activity as measured in cell culture Super TOP-Flash assays (as known it the art and discussed in the example section below).

In an embodiment, the Wnt7 protein is a modulator of the Gpr124- and Reck-dependent Wnt/β-catenin pathway, comprising at its C-terminal end an amino acid addition, having a length of maximum 3 amino acids.

In embodiments, the protein as disclosed herein is an agonist of GPR124/RECK complex, preferably a specific GPR124/RECK complex agonist, wherein said protein is capable of binding to the GPR124/RECK complex, GPR124 protein and/or the RECK protein. In embodiments, said Wnt7 is not able to or has impaired capacity to bind to or to activate Frizzled and/or LRP in the absence of RECK and/or GPR124.

In the present specification, references to any peptides, polypeptides, proteins, or nucleic acids denote the respective peptides, polypeptides, proteins, or nucleic acids as commonly known under the respective designations in the art. More particularly, the references to “Wnt” and in particular to “Wnt7”, to “G-protein coupled receptor 124” (GPR124), “Reversion-inducing cysteine-rich protein with Kazal motifs” (RECK), “Frizzled” (FZD), or “lipoprotein receptor-related protein” (LRP) denote the respective peptides, polypeptides, proteins or nucleic acids, as apparent from the context, as commonly known under said designations in the art.

In certain embodiments, one or more and preferably all of Wnt7, GPR124, RECK, FZD and LRP as employed herein is or are of animal origin, preferably warm-blooded animal origin, more preferably vertebrate origin, yet more preferably mammalian origin, including human origin and non-human mammalian origin, still more preferably human origin.

A skilled person can appreciate that any sequences represented in sequence databases or in the present specification may be precursors of the respective peptides, polypeptides, proteins, or nucleic acids and may include parts that are processed away from mature molecules; or may represent sequences that are of mature molecules while also the precursor sequences are considered part of the present concept.

In embodiments, the Wnt7a/b protein as described herein is a wild-type Wnt7a or Wnt7b comprising at its C-terminal end one or more amino acid additions.

In embodiments, the Wnt7a/b protein as described herein is of animal origin, preferably warm-blooded animal origin, more preferably vertebrate origin, yet more preferably mammalian origin, including human origin and nonhuman mammalian origin, still more preferably mouse or human origin. In another embodiment, the Wnt7a/b protein is of synthetic origin.

SEQ ID NO: 1 and SEQ ID NO: 2 correspond to the mature amino acid sequences of human or mouse Wnt7a and Wnt7b protein sequences respectively.

The amino acid sequence corresponding to SEQ ID NO: 1 (mature human or mouse Wnt7a) is:

LGASIICNKIPGLAPRQRAICQSRPDAIIVIGEGSQMGLDECQFQFRNGR
WNCSALGERTVFGKELKVGSREAAFTYAIIAAGVAHAITAACTQGNLSDC
GCDKEKQGQYHRDEGWKWGGCSADIRYGIGFAKVFVDAREIKQNARTLMN
LHNNEAGRKILEENMKLECKCHGVSGSCTTKTCWTTLPQFRELGYVLKDK
YNEAVHVEPVRASRNKRPTFLKIKKPLSYRKPMDTDLVYIEKSPNYCEED
PVTGSVGTQGRACNKTAPQASGCDLMCCGRGYNTHQYARVWQCNCKFHWC
CYVKCNTCSERTEMYTCK

The amino acid sequence corresponding to SEQ ID NO: 2 (mature human or mouse Wnt7b) is:

LGANIICNKIPGLAPRQRAICQSRPDAIIVIGEGAQMGINECQYQFRFGR
WNCSALGEKTVFGQELRVGSREAAFTYAITAAGVAHAVTAACSQGNLSNC
GCDREKQGYYNQAEGWKWGGCSADVRYGIDFSRRFVDAREIKKNARRLMN
LHNNEAGRKVLEDRMQLECKCHGVSGSCTTKTCWTTLPKFREVGHLLKEK
YNAAVQVEVVRASRLRQPTFLRIKQLRSYQKPMETDLVYIEKSPNYCEED
AATGSVGTQGRLCNRTSPGADGCDTMCCGRGYNTHQYTKVWQCNCKFHWC
CFVKCNTCSERTEVFTCK

In some embodiments Wnt7a/b protein as disclosed here have a sequence according to SEQ ID NO: 1 having at its C-terminus an amino acid addition, having a length of maximum 3 amino acids.

In particular embodiments, the Wnt7a/b protein as disclosed herein comprises amino acid sequence of human or mouse Wnt7a with one to three amino acid additions to its C-terminus.

In some embodiments, Wnt7a/b protein as disclosed here has a sequence according to SEQ ID NO: 2 having at its C-terminus an amino acid addition, having a length of maximum 3 amino acids.

In particular embodiments, the Wnt7a/b protein as disclosed herein comprises amino acid sequence of human or mouse Wnt7b with one to three amino acid additions to its C-terminus.

In embodiments, the C-terminal end amino acid addition of the Wnt7a/b protein as disclosed herein can have a length in a range of 1 to 3 amino acids, preferably of 1 to 2 amino acids, more preferably 1 amino acid.

In an embodiment, said amino acid addition comprises amino acids selected from the 22 alpha-amino acids known to build proteins (so-called proteinogenic amino acids).

According to embodiments, the amino acids of the C-terminal amino acid addition as disclosed herein can be chosen from any natural or non-natural amino acids or the combination thereof. The amino acids of said amino acid addition are preferably chosen from natural amino acids, more preferably chosen from methionine, tryptophan, threonine, isoleucine, phenylalanine, valine, leucine, tyrosine, or a combination thereof.

In other embodiments, C-terminal amino acid addition can have a length of one amino acid. While any amino acid addition is possible, said one amino acid addition is in a preferred embodiment a methionine, a tryptophan, a threonine, isoleucine, phenylalanine, valine, leucine, tyrosine, more preferably methionine, a tryptophan, a threonine, even more preferably a methionine.

As indicated, the term “amino acid addition” as used throughout this specification means an entity of one to three amino acids which can be added to C-terminal end of a protein or (poly)peptide, preferably via peptide bonds. The term may be used to refer to only one, two, or three amino acids. If longer than one amino acid, the amino acids of the amino acid addition are preferably joined together by peptide bonds.

Therefore, this Wnt7 protein comprises a sequence according to SEQ ID NO: 1 or SEQ ID NO: 2, with further at its C-terminus an amino acid addition, having a length of 1 to 3 amino acids, or a length as described in any of the embodiments, said Wnt7 protein has a sequence length similar to SEQ ID NO: 1 or 2 plus 1 to 3 additional amino acids of the amino acid addition.

SEQ ID NO: 1 and 2 each have a length of 348 amino acids, and therefore, in an embodiment, the Wnt7 protein preferably has a length of more than 348 amino acids, preferably 349 amino acids to 351 amino acids, more preferably 349 amino acids to 350 amino acids, most preferably 349 amino acids.

In embodiments, the Wnt7a/b protein may bind RECK protein, optionally to the cysteine knot 4 (CK4) region, to the CK5 region, or to the CK4 and CK5 regions, of RECK protein.

RECK is composed of five N-terminal cysteine-knot (CK) motifs or regions (i.e. CK1, CK2, CK3, CK4 and CK5), a cysteine-rich domain (CRD) and three Kazal motifs preceding a Glycosylphosphatidylinositol (GPI)-anchor site. The CK motifs, the CRD and the Kazal motifs are located extracellularly. Accordingly, in embodiments, the protein capable of binding to the RECK protein as disclosed herein may bind the CK1 motif, CK2 motif, CK3 motif, CK4 motif, CK5 motif, CRD, and/or one or more of the Kazal motifs of the RECK protein, preferably said protein binds to the CK4 and/or CK5 regions of the RECK protein.

In embodiments, the protein disclosed herein can concurrently bind to Frizzled (FZD), LRP and RECK. The protein can bind to the cysteine-rich domain (CRD) of the Frizzled protein. The protein can bind to the LRP protein, for example the protein can bind to the extracellular domain of the LRP protein, to the DKK-binding site and/or the Wnt-binding site of the LRP protein. In more particular embodiments, the protein disclosed herein is capable of binding to the DKK1-binding site of the LRP5 and/or LRP6 protein.

In particular embodiments, the protein as disclosed herein is capable of binding to β-propeller-EGF-like domains 1 and 2 (P1E1P2E2) and/or β-propeller-EGF-like domains 3 and 4 (P3E3P4E4) of the LRP protein.

In particular embodiments, the protein as disclosed herein is capable of concurrently binding to Frizzled and/or LRP proteins in addition to the GPR124 and/or the RECK proteins.

Accordingly, in particular embodiments, the Wnt7a/b protein as disclosed herein is able to bind to Frizzled and LRP proteins, preferably at a cell membrane, in the presence of RECK and GPR124, but is not able to bind to said proteins in the absence of RECK and/or GPR124.

In particular embodiments, the protein as disclosed herein is capable of binding to one or more different Frizzled proteins, such as one or more Frizzled proteins selected from the group consisting of Fzd 1, Fzd2, Fzd3, Fzd4, Fzd5, Fzd6, Fzd7, Fzd8, Fzd9, and Fzd10. Preferably, the compound as disclosed herein is capable of specifically binding to at least Fzd4, and optionally to one or more other Fzd; or capable of specifically binding to Fzd4 substantially to the exclusion of other Fzd. Fzd4 is believed to be the dominant Fzd family member in endothelial cells of the central nervous system. More preferably, the compound as disclosed herein is capable of specifically binding to human Fzd4. The compound as disclosed herein may be selective for the one or more preferred Frizzled proteins, for example having specificity for the one or more preferred Frizzled proteins of at least 5-fold, at least 10-fold, at least 25-fold, at least 50-fold, at least 100-fold, at least 1000-fold, at least 1×10⁴-fold, or at least 1×10⁵-fold, compared to other non-preferred Frizzled proteins.

In particular embodiments, the Wnt7 protein as disclosed herein is capable of binding to one or more different LRP proteins involved in Wnt signaling. Preferably, the protein as disclosed herein is capable of binding to LRP5 and/or LRP6, e.g., any one or each of LRP5 and LRP6. More preferably, the protein as disclosed herein is capable of binding to human LRP5 and/or LRP6, e.g., any one or each of human LRP5 and human LRP6. The protein as disclosed herein may be selective for the one or more preferred LRP proteins, for example having a specificity for the one or more preferred LRP proteins of at least 5-fold, at least 10-fold, at least 25-fold, at least 50-fold, at least 100-fold, at least 1000-fold, at least 1×10⁴-fold, or at least 1×10⁵-fold, compared to other non-preferred LRP proteins.

According to embodiments, the protein disclosed herein is not able to bind FZD and/or LPR if GPR124 and/or RECK is absent.

In embodiments, the absence of GPR124 and/or RECK proteins may denote to a cell expressing FZD and/or LRP proteins (FZD/LRP positive) but having no or low expression of GPR124 and/or RECK proteins (GRP124/RECK negative).

In embodiments, GRP124/RECK negative can mean the no or low expression of GPR124 and/or RECK in a cell, no or low presence of GPR124 and/or RECK proteins in a cell and/or no or low presence of GPR124 and/or RECK proteins at a cell surface.

In embodiments, GRP124/RECK positive can mean the expression of GPR124 and/or RECK in a cell, presence of GPR124 and/or RECK proteins in a cell and/or presence of GPR124 and/or RECK proteins at a cell surface.

In embodiments, FZD/LRP positive can mean the expression of FZD and/or LRP in a cell, presence of FZD and/or LRP protein in a cell, and/or presence of FZD and/or LRP proteins at a cell surface.

In particular embodiments, cells expressing GPR124, RECK, FZD and LRP proteins at its plasma membrane is a cell naturally expressing all GPR124, RECK, FZD and LRP proteins at the cell surface, such as a cerebral endothelial cell.

In some embodiments “the presence of RECK and GPR124” may refer to the presence of the RECK and GPR124 proteins at or close to the cell membrane, preferably in close proximity with the Frizzled and LRP proteins. The close mutual proximity can facilitate the formation of the GPR124/RECK/Frizzled/LRP receptor complex under conditions conducive thereto, such as when a Wnt7 protein as taught herein is supplied to a cell. In embodiments, when used in gene or RNA therapy, a Wnt7a/b protein as taught herein could be expressed from the target cell population. On the other hand, the phrase “the absence of RECK and/or GPR124” may refer to lack or non-occurrence of the RECK and/or GPR124 proteins at the cell membrane. The absence of RECK and/or GPR124 at or close to the cell membrane may occur when a cell does not express, translate, or correctly translocate RECK and/or GPR124. The absence of RECK and/or GPR124 does not need to denote the complete absence of the RECK and/or GPR124 polypeptide at the cell membrane, but may for example refer to an amount of RECK and/or GPR124 polypeptide which is not detectable by, or falls below the sensitivity range of, conventional protein detection or quantification assays known by the person skilled in the art, such as for example immunoblotting, immunocytochemistry or immunofluorescence.

In some embodiments, the protein as disclosed here should first bind to RECK and/or GPR124 to be able to bind FZD and/or LRP proteins.

In embodiments, said protein is capable of activating GPR124/RECK/Frizzled/LRP-mediated Wnt signaling, wherein said Wnt7 protein does not activate Frizzled/LRP-mediated Wnt signaling in the absence of RECK and/or GPR124.

In particular embodiments, the capability of the Wnt7 protein to activate GPR124/RECK/Frizzled/LRP-mediated Wnt signaling, but not activate Frizzled/LRP-mediated Wnt signaling in the absence of RECK and/or GPR124, denotes the capability of the protein to activate Wnt signaling in cells positive for GPR124, RECK, FZD and LRP, but not in cells positive for FZD and LRP and negative for GPR124 and/or RECK, wherein the cells positive GPR124, RECK, FZD and LRP and the cells positive for FZD and LRP and negative for GPR124 and/or RECK are otherwise substantially identical.

In particular embodiments, the capability of the Wnt7 protein to activate GPR124/RECK/Frizzled/LRP-mediated Wnt signaling, but not activate Frizzled/LRP-mediated Wnt signaling in the absence of RECK and/or GPR124, denotes the capability of said Wnt7 protein to activate canonical Wnt signaling in the presence of RECK and GPR124, but not in the absence of RECK and/or GPR124.

The capability of activating Wnt signaling refers to the ability of the protein as disclosed herein to mimic, reproduce or approximate the signal transduction effect and/or activity of a natural Wnt ligand binding to FZD and LRP, such as to an FZD/LRP complex.

Activation of Wnt signaling may be suitably determined and/or quantitated by measuring the expression of one or more Wnt target genes, TCF reporter gene expression, beta-catenin stabilization, LRP phosphorylation, and/or translocation of Axin from cytoplasm to cell membrane as known in the art. For instance, activation of Wnt signaling may be suitably determined and/or quantitated by measuring the expression of TCF gene (e.g., by RT-PCR or any other transcript detection method), a primary output of Wnt signaling. For example, a TCF reporter assay (also known as TOP or TOPflash) may be used to assess changes in the transcription of TCF/LEF controlled genes. The TCF reporter assay may be a luciferase reporter assay. Further for example, activation of Wnt signaling may be suitably determined and/or quantitated by measuring the expression of c-myc, n-myc, LEF1, or c-jun. Alternatively, activation of Wnt signaling may be determined by measuring the location, level and/or phosphorylation status of β-catenin. A non-limiting example of such an assay is the “β-Catenin Redistribution Assay” (Thermo Scientific) which provides recombinant U20S cells stably expressing human β-catenin fused to the C-terminus of enhanced green fluorescent protein (EGFP). The assay allows visualization and monitoring of the translocation of a GFP-β-catenin fusion protein from the membrane to the nucleus. Another way of determining activation of Wnt signaling is the visualization of Axin translocation, for example with a GFP-Axin fusion protein.

In particular embodiments, the Wnt7 protein as disclosed herein may be considered capable of activating (canonical) Wnt signaling if the compound enhances Wnt/β-catenin signaling at least 10-fold more, at least 20-fold more, at least 30-fold more, at least 40-fold more, at least 50-fold more, at least 100-fold more, at least 250-fold more, at least 500-fold more, at least 750-fold more, at least 1000-fold more, at least 1×10⁴-fold more, or at least 1×10⁵-fold more compared to Wnt/β-catenin signaling baseline or background induced by a neutral substance or negative control, for example as measured in an assay as described elsewhere herein.

In particular embodiments, the Wnt7 protein as disclosed herein may be considered to not activate (canonical) Wnt signaling if the compound enhances Wnt/β-catenin signaling less than 10-fold more, such as particularly at most 5-fold more or at most 2.5-fold more, or if the compound does not enhance or even reduces (e.g., 2-fold less or 5-fold less or 10-30 fold less) Wnt/β-catenin signaling compared to Wnt/β-catenin signaling baseline or background induced by a neutral substance or negative control, for example as measured in an assay as described elsewhere herein.

In particular embodiments, the Wnt7a/b proteins as disclosed herein may be considered to activate the GPR124/RECK/Frizzled/LRP-mediated Wnt signaling (denoted “on-target activity”), but not activate Frizzled/LRP-mediated Wnt in the absence of RECK and/or GPR124 (denoted “off-target activity”), if the on target activity is induced at least 30% and off-target activity is induced less than 20% by said compound.

In embodiments, said Wnt7a/b protein can activate GPR124/RECK/Frizzled/LRP-mediated Wnt signaling more than 30%, more than 40%, more than 50%, more than 60%, more than 70%, more than 80%, more than 90%, more than 95%, more than 99% and all ranges and subranges therein, preferably more than 70%. In embodiments, said Wnt7 protein can activate Frizzled/LRP-mediated Wnt in the absence of RECK and/or GPR124 less than 20%, less than 18%, less than 16%, less than 14%, less than 12%, less than 10%, less than 8%, less than 6%, less than 4%, less than 2%, less than 1% and all ranges and subranges therein, preferable less than 10%. Before comparing the GPR124/RECK/Frizzled/LRP mediated Wnt signaling activity (denoted “activity 1” in this paragraph) and the Frizzled/LRP-mediated Wnt signaling activity in the absence of RECK and/or GPR124 (denoted “activity 2” in this paragraph), activity 1 and activity 2 induced by the compound may be normalized to activity 1 and activity 2 induced by wild-type Wnt7a, respectively, the latter for example set to represent 100% activity.

In an embodiment, the protein of the present invention acts by Wnt7-specific RECK/GPR124/Frizzled/LRP-mediated signaling, in which Wnt7 (Wnt7a or Wnt7b) can bind specifically to RECK in a FZD-independent manner and GPR124, a RECK binding partner, can bridge RECK-bound Wnt7 to the FZD/LRP complex via intracellular DVL scaffolds, thereby can allow the assembly of Wnt7-ligand specific RECK/GPR124/FZD/LRP signalosomes and activating canonical Wnt signaling.

The skilled person shall further appreciate that a GPR124/RECK/FZD/LRP receptor Complex, as envisaged herein, may include further component(s), which may or need not functionally modulate the complex. For example, the complex may include Dishevelled (Dvl) forming intracellular scaffolds capable of bridging GPR124 and Frizzled.

According to embodiments, the Wnt7 protein of the disclosed invention may induce the heteromerization of Frizzled and LRP proteins at a cell membrane in the presence of RECK and GPR124, but not in the absence of RECK and/or GPR124.

Activation of the Wnt signaling pathway may occur by promoting the close association or mutual proximity of the Frizzled and LRP proteins at the cell membrane, thereby forming membrane-associated hetero-oligomers comprising the Frizzled and LRP proteins (ligand-driven formation of the Frizzled-LRP hetero-oligomer).

In some tissues expressing GPR124 and/or RECK such as cerebral ECs, a second Wnt7a/b-specific receptor complex is formed to activate Wnt/β-catenin signaling. RECK stabilizes the ligand in a signaling competent lipophilic conformation and delivers it to Frizzled receptors via GPR124. Thereby, RECK and GPR124 synergistically stimulate Wnt7a/b specific responses by assembling higher-order GPR124/RECK/FZD/LRP5/6 complex.

According to embodiments, the Wnt7 protein of the disclosed invention may induce the heteromerization of Frizzled and LRP proteins in the presence of RECK and/or GPR124. The Wnt7 protein of disclosed invention preferably first binds to the RECK protein to induce the heteromerization of Frizzled and LRP protein, more preferably first binds to the RECK/GPR124 complex to induce the heteromerization of Frizzled and LRP protein. According to embodiments, the Wnt7 protein of the disclosed invention may not be able to induce heteromerization of Frizzled and LRP proteins in the absence of RECK and/or GPR124, more specifically said Wnt7 protein is not able to induce heteromerization of Frizzled and LRP proteins when it is not bound to RECK and/or when it is not bound to GPR124 protein and/or when it is not bound to GPR124/RECK complex.

In particular embodiments, the protein of the invention as disclosed herein has at least 35%, preferably at least 40%, at least 50%, at least 60%, at least 70%, at least 80%, at least 90%, at least 95%, at least 99%, preferably 100%, of the GPR124/RECK/Frizzled/LRP-mediated Wnt signaling activity of the full-length wild-type Wnt7 protein. In preferred embodiments, the protein as disclosed herein has at least 70% of the GPR124/RECK/Frizzled/LRP-mediated Wnt signaling activity of the full-length wild-type Wnt7 protein.

In particular embodiments, the protein of the invention as disclosed herein may activate GPR124/RECK/Frizzled/LRP-mediated Wnt signaling in the presence of RECK and/or GPR124 more than 30% of the activation by full-length wild-type Wnt7 protein. For example, more than 30%, more than 40%, more than 50%, more than 60%, more than 70%, more than 80%, more than 90%, more than 95%, more than 99% and all ranges and subranges therein.

In particular embodiments, the Wnt7a/b protein of the invention may activate Wnt signaling in the absence of RECK and/or GPR124 less than 20% of the activation by full-length wild-type Wnt7 protein. For example, less than 18%, less than 16%, less than 14%, less than 12%, less than 10%, less than 8%, less than 6%, less than 4%, less than 2%, less than 1% and all ranges and subranges therein.

In a further aspect, the invention relates to a nucleic acid encoding the Wnt7 protein as disclosed herein. Said nucleic acid may be inserted into a nucleic acid expression cassette and/or vector, as is known in the art.

Accordingly, a further aspect relates to a nucleic acid expression cassette comprising the nucleic acid encoding the protein as disclosed herein, operably linked to a promoter and/or transcriptional and translational regulatory signals.

Preferably, the nucleic acid expression cassette may comprise one or more open reading frames (ORF) encoding said one or more proteins, polypeptides or peptides.

The precise nature of transcriptional and translational regulatory sequences or elements required for expression may vary between expression environments, but typically include a transcription terminator, and optionally an enhancer.

In particular embodiments, the nucleic acid expression cassette comprises the nucleic acid encoding the protein as disclosed herein, operably linked to one or more promoters, enhancers, ORFs and/or transcription terminators.

Also disclosed herein is a nucleic acid encoding the protein as disclosed herein, a vector comprising the nucleic acid encoding the protein as disclosed herein, or the nucleic acid expression cassette as disclosed herein, such as a viral vector.

Nucleic acid can be deoxyribonucleic acid (DNA) and/or ribonucleic acid (RNA). The RNA is preferably an mRNA.

Factors of importance in selecting a particular vector include inter alia: choice of recipient cell, ease with which recipient cells that contain the vector may be recognized and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A nucleic acid can be introduced into a cell in the form of an expression vector such as a plasmid, phage, transposon, cosmid or virus particle. The recombinant nucleic acid can be maintained extra-chromosomally or it can be integrated into the cell's chromosomal DNA. Expression vectors can contain selection marker genes encoding proteins required for cell viability under selected conditions (e.g., URA3, which encodes an enzyme necessary for uracil biosynthesis, or LEU2, which encodes an enzyme required for leucine biosynthesis, or TRP1, which encodes an enzyme required for tryptophan biosynthesis) to permit detection and/or selection of those cells transformed with the desired nucleic acids. Expression vectors can also include an autonomous replication sequence (ARS). The ARS may comprise a centromere (CEN) and an origin of replication (ORI). For example, the ARS may be ARS18 or ARS68.

Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the cell species to be transformed. A nucleotide sequence containing a nucleic acid of interest for expression is inserted in this vector between the first and second insertable DNA fragments, whether before or after the marker gene. Integrative vectors can be linearized prior to transformation to facilitate the integration of the nucleotide sequence of interest into the cell genome. Prior to introducing the vectors into a cell of interest, the vectors can be grown (e.g., amplified) in bacterial cells such as Escherichia coli (E. coli). The vector DNA can be isolated from bacterial cells by any of the methods known in the art, which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

In other particular embodiments, the vector comprising the nucleic acid as described herein is a viral vector, preferably a viral vector specifically directed towards the central and/or peripheral nervous system (e.g., a brain-specific viral vector). In further particular embodiments, the viral vector is a central nervous system (CNS) neuron-specific adeno-associated virus serotype 9 (AAV9) mutant.

In preferred embodiments, the viral vector is a blood brain barrier endothelial cell-specific viral vector. In further preferred embodiments, the viral vector is a blood brain barrier endothelial cell-specific capsid adeno-associated virus serotype 2 (AAV2) mutant.

In embodiments the protein, the nucleic acid, the nucleic acid expression cassette and/or the vector disclosed herein can be used in a method of activating canonical Wnt signaling in the presence of RECK and GPR124, but not in the absence of RECK and/or GPR124, comprising administering to a mammal, a therapeutically effective amount of at least one of said molecules.

### Pharmaceutical Compositions and Formulations

In some embodiments, an alteration to the wild-type Wnt7 protein as a little as addition of single amino acid is efficient to make a tissue-specific, on-target ligand for Wnt signaling.

In embodiments, the subject matters of the invention activate GPR124/RECK/Frizzled/LRP-mediated Wnt signaling (“on-target”, e.g. in cerebral endothelial cells).

In embodiments, the subject matters of the invention have little to zero effect on the activation of unwanted Frizzled/LRP-mediated Wnt signaling in the absence of RECK and/or GPR124, therefore, resulting in even lower “off-target” activity which is making the subject matters of the invention particularly suitable to target cells and tissues expressing RECK and/or GPR124, such as CNS vascular endothelial cells (ECs) and especially making them suitable for inducing the Wnt signaling in said target cells and/or tissues. Therefore, such proteins, nucleic acids, nucleic acid expression cassettes and/or vectors disclosed herein are highly suitable to use as pharmaceuticals to induce Wnt signaling in specific tissues expressing GPR124 and RECK but not induce other FZD-mediated Wnt signaling.

Disclosed herein are pharmaceutical compositions comprising a therapeutically active amount of a compound chosen from the Wnt7 protein as disclosed herein, the nucleic acid encoding the protein as disclosed herein, the nucleic acid expression cassette as disclosed herein, or the vector as disclosed herein with an optional pharmaceutically suitable carrier, diluent, excipient, or adjuvant.

In embodiments, the active principle of the pharmaceutical composition may comprise a protein as described herein. Such may be suitably obtained through expression by host cells or host organisms, transformed with an expression construct encoding and configured for expression of said protein in said host cells or host organisms, followed by purification of the protein.

Hence, a further aspect provides a host cell comprising the nucleic acid, nucleic acid expression cassette or vector as taught herein.

In certain embodiments, the host cell may be a bacterial cell, a yeast cell, an animal cell, or a mammalian cell. The Wnt7 protein may be isolated from such cells using any suitable method known to the skilled person.

In embodiments, the pharmaceutical composition as disclosed herein can further comprise a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media oragent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

In particular embodiments, the compounds of the pharmaceutical composition and/or the pharmaceutical composition is provided in a liposome or lipid nanoparticle.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal, intrathecal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending on the specific needs of a given application.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions.

For example, for oral or nasal aerosol or inhalation administration, pharmaceutical compositions may be formulated for administration in the form of aerosols or sprays are, for example, solutions, suspensions or emulsions of the compound as taught herein or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

Examples of carriers for administration via mucosal surfaces depend upon the particular route, e.g., oral, sublingual, intranasal, etc. and are generally known in the art.

For example, for parenteral administration, pharmaceutical compositions may be advantageously formulated as solutions, suspensions or emulsions with suitable solvents, diluents, solubilisers or emulsifiers, etc. as generally known in the art. Injectable solutions or suspensions may be formulated according to known art, using suitable non-toxic, parenterally-acceptable diluents or solvents. The compound and pharmaceutically acceptable salts thereof of the invention can also be lyophilised and the lyophilisates obtained used, for example, for the production of injection or infusion preparations.

Where aqueous formulations are preferred, such may comprise one or more surfactants. For example, the composition can be in the form of a micellar dispersion comprising at least one suitable surfactant, e.g., a phospholipid surfactant. Various types of phospholipids are known in the art. Typically, a surfactant: active substance molar ratio in an aqueous formulation will be from about 10:1 to about 1:10, more typically from about 5:1 to about 1:5, however any effective amount of surfactant may be used in an aqueous formulation to best suit the specific objectives of interest.

In preferred embodiments, the pharmaceutical composition comprising the protein or nucleic acid encoding the protein, as taught herein is administered parenterally. More preferably, the pharmaceutical composition as taught herein is administered intravenously, for example by infusion, or intrathecally.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

In embodiments the pharmaceutical composition can be used in a method of activating canonical Wnt signaling in the presence of RECK and GPR124, but not in the absence of RECK and/or GPR124, comprising administering to a mammal, a therapeutically effective amount of at least one of said molecules.

In particular embodiments, the compounds as disclosed herein are soluble in water. For example, the solubility of the compound can be increased by using a Frizzled-specific antibody or fragment thereof, instead of a palmitoylated Frizzled-binding domain of a Wnt protein.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

It will be obvious for a skilled person that the aspects as described in “molecules” section above for the determination, analysis and quantification of activation of Wnt signaling suitable here as well.

### Methods of Treatment and Use of Compounds or Compositions

Also disclosed herein are methods of treatment. More particularly, disclosed herein are proteins, nucleic acids, expression cassettes, vectors (compounds hereafter) and/or pharmaceutical compositions as described herein for use as a human or veterinary medicament.

The compounds and pharmaceutical composition disclosed herein allow to provide inter alia novel agonists or modulators capable of stimulating Wnt/β-catenin signaling in cerebral endothelial cells substantially without cross-reactivity with other Frizzled pathways, and useful as therapeutics, particularly for neurovascular disorders or central nervous system (CNS) disorders comprising neurovascular dysfunction.

In particular embodiments, said compounds and pharmaceutical compositions, as disclosed herein, are also suitable to be used in the treatment or prevention of a disorder presenting an abnormal Wnt signaling in RECK and/or GPR124 expressing cells, tissues and/or organs.

In particular embodiments, said neurovascular disorder is selected from the group consisting of ischemic stroke, hemorrhagic stroke, ischemia/reperfusion injury, brain aneurysms, arteriovenous malformations (AVMs), cavernous malformations, vasculitis, cerebral hemorrhage, subarachnoid hemorrhage, spinal vascular malformations, carotid artery stenosis, Moyamoya disease and intracranial atherosclerosis, and retinal vascular disorders, including but not limited to Norrie disease, familial exudative vitreoretinopathy, osteoporosis-pseudoglioma syndrome, diabetic retinopathy and macular degeneration and combinations thereof.

In particular embodiments, said CNS disorder comprising neurovascular dysfunction is selected from the group consisting of multiple sclerosis, ischemic stroke, brain cancer, glioblastoma, human monogenic neurological disorders (e.g. SLC2A1), epilepsy, neurodegenerative disorders, dementia, vascular dementia, HIV-1-associated dementia, Alzheimer's disease, Parkinson's disease, Huntington disease, amyotrophic lateral sclerosis, infectious brain diseases, traumatic brain injuries, migraine, chronic traumatic encephalopathy, neuroinflammation, neurocovid, and combinations thereof.

‘Neuroinflammation’ can refer to a neurovascular inflammation. Further, neuroinflammation can be triggered by different factors. For example, in non-limiting embodiments neuroinflammation is caused by an injury such as traumatic brain injury and/or spinal cord injury, an infection such as viral or bacterial or fungal infection, exposure to a toxin or due to toxic metabolites, neurodegenerative disease, autoimmune disease, smoking or passive smoking, aging or any combination thereof.

Neuroinflammation can be caused by an infectious disease. Said infection may be of bacterial, fungal, parasitic or viral origin. In some instances, the infectious disease is a viral infection. In certain instances, the viral infection comprises infection by one or more of Herpesviridae (Herpes Zoster Virus (VZV) infection, Herpes Simplex Virus (HSV-1 or HSV-2), cytomegalovirus (CMV)), Paramoxyviridae, coronaviruses (SARS-CoV-1 or MERS-COV or SARS-COV-2, HCOVNL63, HCOV-229E, HCoV-OC43, or HKU1 or a variant thereof), influenza viruses (influenza virus A, group 1 (H1N1), group 2 (H2N2), group 3 (H3N2), group 5 (H5N1, H5N2, H5N8) or group 7 (H7N7, H7N9), influenza B, influenza C), Zika virus, Japanese encephalitis virus, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV).

Neuroinflammation can be associated with lung condition such as, acute lung injury (ALI) and/or acute respiratory distress syndrome (ARDS), possibly associated with or arising from ventilator use, viral infection, sepsis, or systemic bacterial infections in a subject in need thereof have been established. Said lung condition can be caused by an infectious disease caused by bacterial, fungal, parasitic or viral infection. Preferably said infection is an infection causing respiratory problems or is a respiratory tract infection. Preferably said infection is a viral infection.

Neuroinflammation can be associated with a bacterial infection. Such inflammation is mostly caused by blood-borne bacteria that cross the blood-brain barrier (BBB) and finally invade the brain parenchyma. Pathogens such as Streptococcus pneumoniae, Neisseria meningitidis, and Haemophilus influenzae are the main etiological causes of bacterial meningitis.

In the present context, prevention and/or treatment of neuroinflammation may include encephalitis. In certain instances, the encephalitis is viral encephalitis, such as COVID-19 associated encephalitis. Neuroinflammation may also be related to inflammation of the optic nerve. Neuroinflammation may also be related to acute disseminated encephalomyelitis, such as COVID-19 associated acute disseminated encephalomyelitis. Neuroinflammation may also be related to inflammation of the vitreous retinal ganglion cells.

In embodiments said compounds and compositions are suitable particularly suitable to be administered to a patient prone to develop or suffering from such disorder.

In particular embodiments, the pharmaceutical composition for use comprising the compound, the nucleic acid encoding the compound, or the nucleic acid expression cassette comprising the nucleic acid as taught herein is used in gene and/or RNA therapy, in particular in blood brain barrier endothelial cell-directed gene therapy.

Accordingly, also provided herein is a method for gene therapy, in particular central and/or peripheral nervous system-directed gene therapy, in a subject in need of said gene therapy comprising: introducing in the subject, in particular in the central and/or peripheral nervous system of the subject, a nucleic acid expression cassette or a vector as described herein; and expressing a therapeutically effective amount of the compound encoded by the nucleic acid as taught herein in the subject, in particular the central and/or peripheral nervous system of the subject.

In particular embodiments, the Wnt7 protein or the nucleic acid encoding said protein as taught herein is used in mRNA therapy, in particular in blood brain barrier endothelial cell-directed mRNA therapy.

Accordingly, also provided herein is a method for RNA therapy, preferably mRNA therapy, in particular central and/or peripheral nervous system-directed mRNA therapy, in a subject in need of said mRNA therapy comprising: introducing in the subject, in particular in the central and/or peripheral nervous system of the subject, a nucleic acid encoding the compound as taught therein; and expressing a therapeutically effective amount of the compound encoded by the nucleic acid as taught herein in the subject, in particular the central and/or peripheral nervous system of the subject.

An advantage of the use of RNAs therapy, it is generally believed that RNAs do not integrate into the genome and therefore do not have the risk of insertional mutagenesis.

Any known methods of introducing nucleic acids into animal cells may be used herein. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors (i.e. derived from lentivirus, adeno-associated virus (AAV), adenovirus, retrovirus or antiviruses), electroporation, and the like. Other techniques or methods which are suitable for delivering nucleic acid (NA) molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable microspheres. Also envisaged is encapsulation of NA or providing NA in various types of liposomes (immunoliposomes, PEGylated (immuno) liposomes), cationic lipids and polymers, nanoparticles or dendrimers, poly (lactic-Co-Glycolic Acid) polymeric microspheres, implantable drug-releasing biodegradable microspheres, etc.; and co-injection of NA with protective agent like the nuclease inhibitor aurintricarboxylic acid. It shall be clear that also a combination of different above-mentioned delivery modes or methods may be used.

In particular embodiments, the compound is provided in carriers, such as liposomes, lipid nanoparticles, nanostructured lipid carriers, nanoemulsions, polymer nanoparticles, polymer micelle or dendrimers. In a preferred embodiment, said carriers are liposomes or lipid nanoparticles (LNPs).

In an embodiment, said carriers are lipid-based carriers. Said lipid-based carriers comprise one or more lipids. The one or more lipids can be in solid and/or liquid form. Said lipid-based carriers may be LNPs, lipoplexes, liposomes, phospholipid micelles, solid lipid nanoparticles, nanostructured lipid carriers or nano-emulsions. Lipid-based carriers useful according to the invention include, for example, cationic lipids, liposomes, in particular cationic liposomes, and micelles, and nanoparticles. Cationic lipids may form complexes with negatively charged nucleic acids. Any cationic lipid may be used according to the invention. Liposomes are phospholipid and cholesterol self-assembled bilayer membranes that enclose an aqueous core, where hydrophilic molecules can be incorporated. Hydrophobic compounds can also be incorporated in the lipid bilayer. Liposomes can be classified in (i) small unilamellar vesicles (SUVs); (ii) large unilamellar vesicles (LUVs) and (iii) multilamellar vesicles (MLVs), according to their size and lamellarity. Solid lipid nanoparticles (SLNs) have a spherical shape with an average diameter of 10-1000 nm. They are used as a colloidal NP drug delivery system in which lipid drug carrier solidifies at room temperature as well as at body temperature. Different solid lipids can be exploited to produce SLNs, such as, tripalmitin, cetyl alcohol, cetyl palmitate, glyceryl monostearate, trimyristin, tristearin, stearic acid, etc. SLNs comprise of solid lipid, such as triglycerides, fatty acids, waxes, partial glycerides, and polyethylene glycosylated lipid; emulsifiers, such as polysorbates, poloxamer and lecithin; and water. Nanostructured lipid carriers (NLC), comprise a blend of solid and liquid lipids which results in a partially crystallized lipid system and many have advantages such as enhanced drug loading capacity, drug release modulation flexibility and improved stability.

In an embodiment, said lipid-based carrier is a lipid nanoparticle. Solid lipid nanoparticles (SLNs, SLNPs), or lipid nanoparticles (LNPs), are nanoparticles composed of lipids that are suited to be used as a drug delivery vehicle for drug compounds, especially polynucleotides such as RNA or DNA.

Compounds such the Wnt7a/b proteins, nucleic acids, expression cassettes, vectors (compounds hereafter) as disclosed herein and compositions detailed herein, such as a pharmaceutical composition containing any of the said compound provided herein or a salt thereof and a pharmaceutically acceptable carrier or excipient, may be used in methods of administration and treatment as provided herein.

In particular embodiments, the Wnt7a/b proteins, nucleic acids, expression cassettes, vectors and/or the pharmaceutical composition as taught herein is administered to the subject by the injection (e.g., intravenously) or transplantation of allogeneic cells transformed with the vector comprising the nucleic acid or the nucleic acid expression cassette as taught herein. When administered, the injected or transplanted allogenic cells will transcribe and translate the nucleic acid encoding the compound as taught herein in vivo.

The dosage or amount of the compound, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the compound described in any of the embodiments above. In order to optimize therapeutic efficacy, the compound as taught herein can be first administered at different dosing regimens. Typically, levels of the compound in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the Wnt7a/b protein, nucleic acid encoding said Wnt7a/b protein or pharmaceutical composition for use of the invention as described in any of the embodiments, comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While Wnt7a/b protein, nucleic acid encoding said Wnt7a/b protein and/or pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosages for use in appropriate subjects. The dosage of such Wnt7a/b protein, nucleic acid encoding said Wnt7a/b protein lies generally within a range of circulating concentrations that include the ED50 with little or no toxicity. The dosage may vary within this range depending upon the dosage form employed and the route of administration utilized. For a Wnt7a/b protein, nucleic acid encoding said Wnt7a/b protein used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

In particular embodiments, the Wnt7a/b protein or nucleic acid encoding said Wnt7a/b protein as taught herein is administered using a sustained delivery system, such as a (partly) implanted sustained delivery system. Skilled person will understand that such a sustained delivery system may comprise a reservoir for holding the compound as taught herein, a pump and infusion means (e.g., a tubing system). For example, the sustained delivery system may be a mini-osmotic pump system implanted in the brain.

In particular embodiment, the Wnt7a/b protein or nucleic acid encoding said Wnt7a/b protein as disclosed herein is the main or only active ingredient of the pharmaceutical composition.

In another embodiment, the Wnt7a/b protein or nucleic acid encoding said Wnt7a/b protein or the pharmaceutical composition for use as described in any of the embodiments is combined with a second therapy, preferably chosen from surgery, chemotherapy, radiotherapy or immunotherapy.

A further aspect relates to the pharmaceutical composition comprising the compound as disclosed herein, the nucleic acid encoding the compound as disclosed herein the nucleic acid expression cassette as disclosed herein or the vector as disclosed herein, for use as a medicament.

A further aspect relates to the pharmaceutical composition comprising the Wnt7a/b protein as disclosed herein, the nucleic acid encoding the Wnt7a/b protein as disclosed herein, the nucleic acid expression cassette as disclosed herein, or the vector as disclosed herein, for use in the prevention or treatment of a neurovascular disorder, a central nervous system (CNS) disorder comprising neurovascular dysfunction.

In particular embodiments, neurovascular disorders can be selected from the group consisting of ischemic stroke, hemorrhagic stroke, ischemia/reperfusion injury, brain aneurysms, arteriovenous malformations (AVMs), cavernous malformations, vasculitis, cerebral hemorrhage, subarachnoid hemorrhage, spinal vascular malformations, carotid artery stenosis, Moyamoya disease, intracranial atherosclerosis, retinal vascular disorders, including but not limited to Norrie disease, familial exudative vitreoretinopathy, osteoporosis-pseudoglioma syndrome, diabetic retinopathy and macular degeneration and combinations thereof.

In an embodiment, said neurovascular disorder is retinopathy, wherein said retinopathy is a retinal vascular disease. In an embodiment, said retinal vascular disease is caused by inhibition of vascular development. In an embodiment, said retinopathy is caused by excessive angiogenesis. In an embodiment, said retinal vascular disease is selected from the group consisting of: familiar exudative vitreoretionopathy (FEVR), exudative vitreoretinopathy, Norrie disease, Nome disease, diabetic retinopathy (DR), age-related macular degeneration (AMD), retinopathy of prematurity (ROP), osteoporosis-pseudoglioma syndrome (OPPG), retinal vein occlusion, and Coats disease.

In particular embodiments, CNS disorder comprising neurovascular dysfunction can be selected from the group consisting of multiple sclerosis, ischemic stroke, brain cancer, glioblastoma, human monogenic neurological disorders (e.g. SLC2A1), epilepsy, neurodegenerative disorders, dementia, vascular dementia, HIV-1-associated dementia, Alzheimer's disease, Parkinson's disease, Huntington disease, amyotrophic lateral sclerosis, infectious brain diseases, traumatic brain injuries, migraine, chronic traumatic encephalopathy, neuroinflammation, neurocovid, COVID-19 and combinations thereof.

‘Neuroinflammation’ can refer to a neurovascular inflammation as described above.

In a further embodiment a method of preventing, reducing in progression or treating a neurovascular disorder or a central nervous system (CNS) disorder comprising neurovascular dysfunction, such as but not limited to the disorders listed above, in a subject in need thereof, comprising administering to a subject a therapeutically active amount of pharmaceutical composition comprising a Wnt7 protein, wherein said Wnt7 protein is a selective agonist of Gpr124- and Reck-dependent Wnt/β-catenin pathway, and comprises at its C-terminal end an amino acid addition, having a length of maximum 3 amino acids, or a nucleic acid encoding for such Wnt7 protein as described in any of the embodiments throughout this specification.

It will be clear that the current invention equally provides methods for the treatment of a neurovascular disorder or a central nervous system (CNS) disorder comprising neurovascular dysfunction, as well as is directed to the use of a therapeutically active amount of pharmaceutical composition comprising Wnt7 protein wherein said protein is a selective agonist of Gpr124- and Reck-dependent Wnt/β-catenin pathway, and comprises at its C-terminal end an amino acid addition, having a length of 1 to 3 amino acids, or a nucleic acid encoding said Wnt7 protein, as described in any of the embodiments throughout this specification for the manufacture of a medicament for the treatment or reduction of progression of a neurovascular disorder or a central nervous system (CNS) disorder comprising neurovascular dysfunction in a subject. Further embodiments are as described above.

One skilled in this art will recognize that the above description is illustrative rather than exhaustive. Indeed, many additional formulations, techniques and pharmaceutically-acceptable excipients and carrier solutions are well-known to those skilled in the art, as is the development of suitable dosing and treatment regimens for using the particular compositions described herein in a variety of treatment regimens.

### Synthesis

The addition of amino acids to the C-terminus of the protein can be done with any method known to the field. For example, the amino acids can be introduced via site-directed mutagenesis to the nucleic acid sequence encoding the protein, via PCR with overhanging primers and/or cloning into vectors. Protein can be made via translation of said vector, or via de novo protein synthesis.

### EXAMPLES AND/OR DESCRIPTION OF FIGURES

The present invention will now be further exemplified with reference to the following examples. The present invention is in no way limited to the given examples or to the embodiments presented in the figures.

### Materials and Methods

### Expression Plasmid Constructs

C-terminal modification (i.e. amino acid addition) of mouse Wnt7a (UniProtKB #P24383), were created using the Q5® Site-Directed Mutagenesis Kit (New England Biolabs®). All modified genes were cloned using pCS2+ vector as template. The identity of all constructs was confirmed by Sanger sequencing.

### Super TOP-Flash Reporter Gene Assay

Dual luciferase assays were performed using Super TOP-Flash HEK293 luciferase reporter cell lines as known in the art.

For ON-target setting, cells were transiently transfected with Renilla luciferase (0.5 ng), Wnt7 variants (20 ng), Fz1 receptor (5 ng), Lrp5 (2.5 ng), Gpr124 (10 ng), Reck (10 ng) in Lipofectamine™ 2000 Transfection Reagent (Invitrogen).

For OFF-target setting, cells were only transfected with plasmids encoding Renilla luciferase (0.5 ng), Wnt7 variants (20 ng), Fz5 receptor (5 ng), and Lrp5 (2.5 ng). The assays were performed 48 h after transfection and the activity was measured. The data shown are pooled from at least three independent experiments each consisting of three technical replicates (mean±standard deviation (SD)).

### Example 1: On-Target and Reduced Off-Target Activity of Wnt7a with Amino Acid Additions Compared to Wt Wnt7a

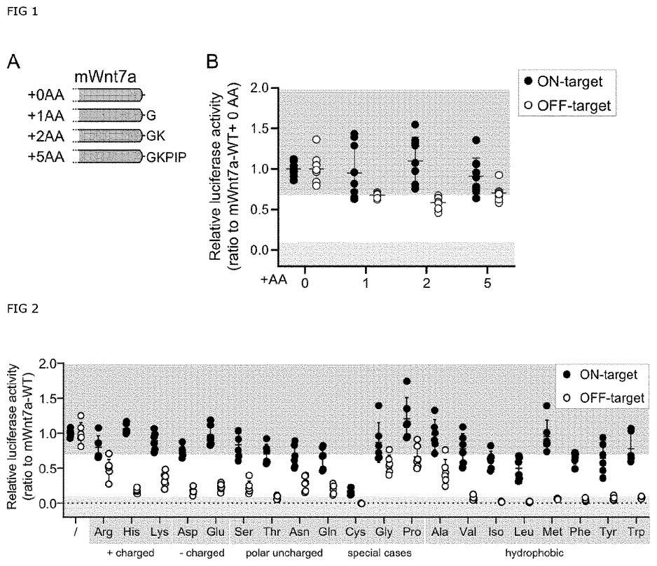

One to five amino acids were added at the C-terminus to unmodified mouse Wnt7a. The resulting constructs were transfected in HEK293 STF cells together with Renilla luciferase, Lrp5 and Fz1 receptor, Gpr124 and Reck constructs (On-target), or with Renilla luciferase, Lrp5 and Fz5 receptor (Off-target) and tested in a dual luciferase assay as described above. Over all, the results showed that the on-target activity of the wt Wnt7 is maintained even after the addition of various length of the amino acid addition (from 1 to 5 aa), while off-target activity is significantly reduced as shown in FIG. 1B.

### Example 2: The Effect of One Amino Acid Addition to the C-Terminus of Wt Wnt7a on “On-Target” Activation and “Off-Target” Activation of the Canonical Wnt Pathway Either with GPR124/RECK (on Target) or without GPR124/RECK (Off-Target)

The effect of the addition of any of the 20 natural amino acids was tested on signaling properties of wild-type Wnt7a. Each of the 20 different amino acids was added to the C-terminus of mouse Wnt7a. The resulting constructs were transfected in HEK293 STF cells together with Renilla luciferase, Lrp5 and Fz5 receptor constructs (off-target) or Lrp5 and Fz1 receptor, Gpr124 and Reck constructs (on-target) and tested in a dual luciferase assay as described above.

Interestingly, the addition of certain amino acids to the C-terminal end of the wild-type mWnt7a protein significantly decreased its off-target activity. The addition of the Methionine residue was particularly interesting because this addition kept an on-target activity similar to mWnt7a without residue addition but lead to an undetectable off-target activity using the in vitro luciferase assay (FIG. 2).

This C-terminal addition of Methionine to the wild-type Wnt7a protein thus yields a promising variant for clinical studies.

It is clear that the method according to the invention, and its applications, are not limited to the presented examples.

The present invention is in no way limited to the embodiments described in the examples and/or shown in the figures. On the contrary, methods according to the present invention may be realized in many different ways without departing from the scope of the invention.

1. A Wnt7 protein characterized in that, said protein comprises at its C-terminal end an amino acid addition, having a length of maximum 3 amino acids.

2. The Wnt7 protein according to claim 1, wherein said Wnt7 protein is an agonist of the Gpr124- and Reck-dependent Wnt/β-catenin pathway.

3. The Wnt7 protein according to claim 1, wherein the sequence of said Wnt7 protein is a sequence according to SEQ ID NO:1 or SEQ ID NO: 2, having at its C-terminus an amino acid addition, having a length of maximum 3 amino acids.

4. The Wnt7 protein according to claim 1, wherein the amino acid addition has a length of 1 to 2 amino acids.

5. The Wnt7 protein according to claim 1, wherein the amino acids of the C-terminal amino acid addition are chosen from Methionine, Tryptophan, Threonine, Isoleucine, Phenylalanine, Valine, Leucine, Tyrosine or a combination thereof.

6. The Wnt7 protein according to claim 1, wherein said amino acid addition has a length of one amino acid, wherein said amino acid is Methionine, Tryptophan, Threonine, Isoleucine, Phenylalanine, Valine, Leucine, or Tyrosine.

7. The Wnt7 protein according to claim 1, having a length of between 349 and 352 amino acids.

8. A nucleic acid encoding for the Wnt7 protein according to claim 1.

9. A nucleic acid expression cassette comprising the nucleic acid according to claim 8, operably linked to a promoter and/or transcriptional and translational regulatory signals.

10. A vector comprising the nucleic acid according to claim 8.

11. A pharmaceutical composition comprising the Wnt7 protein according to claim 1, and a pharmaceutically acceptable carrier.

12. A method comprising administering to an individual in need thereof the pharmaceutical composition according to claim 11 as a medicament.

13. A method comprising administering to an individual in need thereof the nucleic acid according to claim 8, in gene therapy.

14. A method comprising administering to an individual in need thereof the nucleic acid according to claim 8, in RNA therapy.

15. A method comprising administering to an individual in need thereof the pharmaceutical composition according to claim 11 in the prevention or treatment of a neurovascular disorder or a central nervous system (CNS) disorder comprising neurovascular dysfunction; optionally wherein said neurovascular disorder is selected from the group consisting of ischemic stroke, hemorrhagic stroke, ischemia/reperfusion injury, brain aneurysms, arteriovenous malformations (AVMs), cavernous malformations, vasculitis, cerebral hemorrhage, subarachnoid hemorrhage, spinal vascular malformations, carotid artery stenosis, Moyamoya disease intracranial atherosclerosis and combinations thereof, retinopathy wherein said retinopathy is a retinal vascular disease or disorder, and combinations thereof, or said CNS disorder is selected from the group consisting of multiple sclerosis, ischemic stroke, brain cancer, glioblastoma, human monogenic neurological disorders, epilepsy, neurodegenerative disorders, dementia, vascular dementia, HIV-1-associated dementia, Alzheimer's disease, Parkinson's disease, Huntington disease, amyotrophic lateral sclerosis, infectious brain diseases, traumatic brain injuries, migraine, neuroinflammation, COVID-19, and chronic traumatic encephalopathy and combinations thereof.

16. The method according to claim 15, wherein said neurovascular disorder is retinopathy, wherein said retinopathy is retinal vascular disorder which is caused by inhibition of vascular development.

17. The method according to claim 15, wherein said neurovascular disorder is retinopathy, wherein said retinopathy is retinal vascular disorder which is caused by excessive angiogenesis.

18. The Wnt7 protein, the nucleic acid, the nucleic acid expression cassette, the vector, or the pharmaceutical composition, for use method according to claim 15, wherein said neurovascular disorder is retinopathy, wherein said retinopathy is retinal vascular disorder selected from the group consisting of: Norrie disease, Nome disease, familial exudative vitreoretinopathy, diabetic retinopathy and macular degeneration, retinopathy of prematurity (ROP), osteoporosis-pseudoglioma syndrome (OPPG), retinal vein occlusion, and Coats disease.

19. A vector comprising the nucleic acid expression cassette according to claim 9.

20. A method comprising administering to an individual in need thereof the nucleic acid according to claim 8 in blood-brain barrier endothelial cell-directed gene therapy.


SEQUENCE LISTING (4 sequences)

SEQ ID NO: 1 - protein, 318 aa, Homo sapiens
LGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 2 - protein, 318 aa, Homo sapiens
LGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 3 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 4 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK